(19) United States
(12) Patent Application Publication
(10) Pub. No.: US 2013/0302409 A1
(43) Pub. Date: Nov. 14, 2013

(54) METHODS AND COMPOSITIONS FOR CANCER IMMUNOTHERAPY

(71) Applicants: Ephraim Joseph Fuchs, Owings Mills, MD (US); Tarek M. Fahmy, New Haven, CT (US)

(72) Inventors: Ephraim Joseph Fuchs, Owings Mills, MD (US); Tarek M. Fahmy, New Haven, CT (US)

(73) Assignees: YALE UNIVERSITY, New Haven, CT (US); THE JOHNS HOPKINS UNIVERSITY, Baltimore, MD (US)

(21) Appl. No.: 13/810,548

(22) PCT Filed: Jul. 15, 2011
(86) PCT No.: PCT/US11/44134

§ 371 Date: Jul. 29, 2013

Related U.S. Application Data
(60) Provisional application No. 61/364,840, filed on Jul. 16, 2010.

(51) Int. Cl. A61K 39/00; A61K 38/46; A61K 45/06

(52) U.S. Cl. CPC A61K 39/0011; A61K 45/06; A61K 38/46
USPC 424/451; 424/277.1

(57) ABSTRACT

The present invention generally relates to the field of cancer and methods and compositions for cancer immunotherapy. In one embodiment, a method for treating cancer in a patient comprises the steps of (a) administering at or near the cancer site an effective amount of a composition that promotes a therapeutic immune response to the cancer; and (b) ablating the cancer. In another embodiment, a method for treating an abnormal cellular proliferation in a patient comprises the steps of (a) administering at or near the site of the abnormal cellular proliferation an effective amount of a composition that promotes a therapeutic immune response to the abnormal cellular proliferation comprising (i) a polymeric particle; and (ii) optionally one or more therapeutic agents encapsulated in or incorporated on or into the polymeric particle; and (b) ablating the abnormal cellular proliferation.

### CROSS-REFERENCE TO RELATED APPLICATIONS

This application claims the benefit of U.S. Provisional Application No. 61/364,840, filed Jul. 16, 2010; which is incorporated herein by reference in its entirety.

### FIELD OF THE INVENTION

The present invention generally relates to the field of cancer and methods and compositions for cancer immunotherapy.

### BACKGROUND OF THE INVENTION

Surgery, radiation therapy, and chemotherapy have been the standard accepted approaches for treatment of cancers including leukemia, solid tumors, and metastases. Immunotherapy uses the body's immune system, either directly or indirectly, to shrink or eradicate cancer, and has been studied for many years as an adjunct to conventional cancer therapy. It is believed that the human immune system is an untapped resource for cancer therapy and that effective treatment can be developed once the components of the immune system are properly harnessed. As key immunoregulatory molecules and signals of immunity are identified and prepared as therapeutic reagents, the clinical effectiveness of such reagents can be tested using well-known cancer models. Immunotherapeutic strategies include administration of vaccines, activated cells, antibodies, cytokines, chemokines, as well as small molecular inhibitors, anti-sense oligonucleotides, and gene therapy. Although much has been learned about controlling and directing an immune response, there is need for newer and more effective immunotherapeutic approaches to cancer therapy.

### SUMMARY OF THE INVENTION

The present invention generally relates to the field of cancer and methods and compositions for cancer immunotherapy. The present invention is based, at least in part, on the discovery that biodegradable nanoparticles engineered to function as immunological adjuvants stimulate the T cell response to cancer in vivo by activating antigen-presenting cells (APCs) in the tumor microenvironment and by ferrying antigens into APCs for processing and presentation to tumor-reactive T cells. In particular embodiments, intratumoral injection of nanoparticles followed by cryoablation generates a potent, patient-specific, cell-based tumor vaccine for any type of solid malignancy. The nanoparticles synergize with other immune manipulations to overcome mechanisms of tumor-induced tolerance, including the negative influences of regulatory T cells, suppressive APCs, and lack of tumor-specific CD4⁺ effector helper T cells. Antigens emulsified in adjuvants such as alum typically elicit type 2 helper T cell responses and antibody production, but not CD8⁺ T cell responses. In contrast, antigens in nanoparticles are processed and presented for recognition by CD8⁺ T cells, which are critical effectors of anti-tumor immunity. Combination therapies that include nanoparticles can effectively unmask potent, systemic anti-tumor immunity leading to elimination of micrometastases and prevention of relapse of early stage cancers, or regression of macroscopic tumors and prolongation of survival in patients with metastatic cancer.

Accordingly, in one aspect, the present invention provides methods and compositions useful for treating cancer. In one embodiment, a method for treating cancer in a patient comprises the steps of (a) administering at or near the cancer site an effective amount of a composition that promotes a therapeutic immune response to the cancer; and (b) ablating the cancer. In another embodiment, the composition comprises (a) a polymeric particle; and (b) optionally one or more therapeutic agents encapsulated in or incorporated on or into the polymeric particle.

In another aspect, the present invention provides methods and compositions useful treating an abnormal cellular proliferation. In a specific embodiment, a method for treating an abnormal cellular proliferation in a patient comprises the steps of (a) administering at or near the site of the abnormal cellular proliferation an effective amount of a composition that promotes a therapeutic immune response to the abnormal cellular proliferation comprising (i) a polymeric particle; and (ii) optionally one or more therapeutic agents encapsulated in or incorporated on or into the polymeric particle; and (b) ablating the abnormal cellular proliferation. In a more specific embodiment, the one or more therapeutic agents is an antigen preferentially expressed by the abnormally proliferating cell.

The methods of the present invention can further comprise the step of administering an effective amount of an agent that mitigates suppression of anti-tumor immunity to the patient prior to or after administering the composition. In particular embodiments, the agent is selected from the group consisting of alkylating agents, steroids, nucleotide inhibitory drugs, chemotherapeutics, monoclonal antibodies, toxins, and inflammatory reducing agents. In more specific embodiments, the agent is selected from the group consisting of cyclophosphamide, 5-fluorouracil, gemcitabine, doxorubicin, denileukin, diftitox, bevacizumab, and docetaxel.

In certain embodiments, the polymeric particle comprises poly lactide (PLA), polyglycolide (PGA), poly(lactic-co-glycolic acid) (PLGA) or co-polymers thereof. In a specific embodiment, the polymeric particle is PLGA.

In other embodiments, the compositions of the present invention further comprise one or more immunological adjuvants encapsulated in or incorporated on or into the polymeric particle. In a specific embodiment, the immunological adjuvant is a Toll-Like Receptor (TLR) Ligand. In a more specific embodiment, the immunological adjuvant is monophosphoryl lipid A (MPL). In an alternative embodiment, the immunological adjuvant is lipopolysaccharide (LPS). In another embodiment, the immunological adjuvant is a C-Type Lectin Receptor Ligand. In a further embodiment, the immunological adjuvant is a Nucleotide Oligomerization Domain (NOD)-Like Receptor Ligand. The immunological adjuvant can also be a Retinoic Acid-Inducible Gene-I (RIG)-Like Receptor (RLR) Ligand. Alternatively, the immunological adjuvant is a Receptor for Advanced Glycation Endproducts (RAGE) Ligand. In yet another embodiment, the immunological adjuvant is selected from the group consisting of LPS or derivatives thereof, CpG oligos, TLR3 ligands, TLR7 ligands, TLR9 ligands, MPL ligands, and RC529. Indeed, one or more immunological adjuvants can be encapsulated in, incorporated on or into the polymeric particle. For example, a TLR4 ligand and a TLR7 ligand can be encapsulated in or incorporated on or into a polymeric particle.

In a specific aspect, the one or more therapeutic agents is a cancer antigen. In particular embodiments, the one or more therapeutic agents is selected from the group consisting of tumor antigens. CD4⁺ T-cell epitopes, cytokines, chemotherapeutic agents, radionuclides, small molecule signal transduction inhibitors, photothermal antennas, small interfering RNAs, monoclonal antibodies, and immunologic danger signaling molecules. In a specific embodiment, the therapeutic agent is Sipuleucel-T. In such embodiments, the abnormal cellular proliferation is prostate cancer. The therapeutic agent can also be carbonic anhydrase-IX. In such embodiments, the abnormal cellular proliferation is kidney cancer, colon cancer or cervical cancer. In a further embodiment, the therapeutic agent is carcinoembryonic antigen. In these embodiments, the abnormal cellular proliferation is breast cancer, lung cancer or colon cancer.

In the methods of the present invention, the step of ablating the cancer is accomplished by a method selected from the group consisting of cryoablation, thermal ablation, radiotherapy, chemotherapy, radiofrequency ablation, electroporation, alcohol ablation, high intensity focused ultrasound, photodynamic therapy, monoclonal antibodies, and immunotoxins. In a specific embodiment, the step of ablating the cancer is accomplished by cryoblation.

In a more specific embodiment, the present invention provides a method for treating a solid tumor in a patient comprising the steps of (a) administering an effective amount of an agent that mitigates suppression of anti-tumor immunity to the patient; (b) administering at or near the tumor site an effective amount of a composition comprising (i) a polymeric nanoparticle; (ii) one or more TLR ligands, C-Type Lectin Receptor ligands, NOD-Like Receptor Ligands, RLR Ligands, and/or RAGE Ligands encapsulated in or incorporated on or into the nanoparticle; and (iii) one or more tumor antigens encapsulated in the nanoparticle; and (c) applying cryoablation to the solid tumor.

In another embodiment, the present invention provides a method for treating a solid tumor in a patient comprising the steps of (a) administering at or near the tumor site an effective amount of a composition comprising (i) a polymeric nanoparticle; (ii) one or more TLR ligands, C-Type Lectin Receptor ligands, NOD-Like Receptor ligands, RLR ligands, and/or RAGE ligands encapsulated in or incorporated on or into the nanoparticle; and (iii) one or more tumor antigens encapsulated in the nanoparticle; and (b) ablating the solid tumor.

In yet another embodiment, a method for treating a cancer in a patient comprises the steps of (a) administering an effective amount of cyclophosphamide to the patient; (b) administering at or near the tumor site an effective amount of a composition comprising (i) a nanoparticle comprising PLGA: (ii) MPL incorporated on to the nanoparticle; and (iii) one or more tumor antigens encapsulated in the nanoparticle; and (c) ablating the cancer.

### BRIEF DESCRIPTION OF THE FIGURES

FIG. 1 depicts a particle of the present invention. FIG. 1A is a schematic diagram of lipopolysaccharide (LPS)-modified, antigen-encapsulated nanoparticles. FIG. 1B shows a scanning electron micrograph of a nanoparticle.

FIG. 2 illustrates the experimental protocol described in Example 1.

FIG. 3 is a graph showing the results of the administration of cyclophosphamide (Cy or Cytoxan) and a composition of the present invention, followed by cryoablation, as further described in Example 1.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “particle” is a reference to one or more particles, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. DEFINITIONS

The following definitions are used throughout this specification. Other definitions are embedded within the specification for ease of reference.

The term “adjuvant” refers to any substance that assists or modifies the action of a pharmaceutical, including but not limited to immunological adjuvants, which increase and/or diversify the immune response to an antigen. Hence, immunological adjuvants are compounds that are capable of potentiating an immune response to antigens. Immunological adjuvants can potentiate humoral and/or cellular immunity. In some embodiments, immunological adjuvants stimulate an innate immune response. Immunological adjuvants may also be referred to herein as “immunopotentiators.”

As used herein, an “antigen” refers to a molecule containing one or more epitopes (e.g., linear, conformational or both) that elicit an immunological response. The term may be used interchangeably with the term “immunogen.” The term “antigen” can denote both subunit antigens, i.e., antigens which are separate and discrete from a whole organism with which the antigen is associated in nature, as well as killed, attenuated or inactivated bacteria, viruses, parasites or other pathogens or tumor cells. Antibodies such as anti-idiotype antibodies, or fragments thereof, and synthetic peptide mimotopes, which can mimic an antigen or antigenic determinant, are also within the definition of antigen. Similarly, an oligonucleotide or polynucleotide that expresses an immunogenic protein, or antigenic determinant in vivo, such as in nucleic acid immunization applications, is also included in the definition of antigen herein.

As used herein, the term “cancer” means a type of hyperproliferative disease that includes a malignancy characterized by deregulated or uncontrolled cell growth. Cancers of virtually every tissue are known. Examples of cancer include, but are not limited to, carcinoma, lymphoma, blastoma, sarcoma, and leukemia or lymphoid malignancies. More particular examples of such cancers are noted below and include squamous cell cancer (e.g., epithelial squamous cell cancer), lung cancer (including small-cell lung cancer, non-small cell lung cancer, adenocarcinoma of the lung and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer, uterine carcinoma, salivary gland carcinoma, kidney or renal cancer, prostate cancer, thyroid cancer, hepatic carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

The term “cancer,” is encompassed within the scope of the broader term “abnormal cellular proliferation, which can also be referred to as “excessive cellular proliferation or “cellular proliferative disease.” Examples of diseases associated abnormal cellular proliferation include metastatic tumors, malignant tumors, benign tumors, cancers, pre-cancers, hyperplasias, warts, and polyps, as well as non-cancerous conditions such as benign melanomas, benign chondroma, benign prostatic hyperplasia, moles, dysplastic nevi, dysplasia, hyperplasias, and other cellular growths occurring within the epidermal layers. Classes of precancers include acquired small or microscopic precancers, acquired large lesions with nuclear atypia, precursor lesions occurring with inherited hyperplastic syndromes that progress to cancer, and acquired diffuse hyperplasias and diffuse metaplasias. Examples of small or microscopic precancers include HGSIL (high grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma.

As used herein, an “epitope” is that portion of given species (e.g., an antigenic molecule or antigenic complex) that determines its immunological specificity. An epitope is within the scope of the present definition of antigen. Commonly, an epitope is a polypeptide or polysaccharide in a naturally occurring antigen. In artificial antigens, it can be a low molecular weight substance such as an arsanilic acid derivative. Normally, a B-cell epitope will include at least about 5 amino acids but can be as small as 3-4 amino acids. A T-cell epitope, such as a CTL epitope, will typically include at least about 7-9 amino acids, and a helper T-cell epitope will typically include at least about 12-20 amino acids.

An “immunological response” or “immune response” to an antigen or a given species (e.g., an antigenic molecule, a cancer cell, an abnormal cellular proliferation, etc.) is the development in a subject of a humoral and/or a cellular immune response to molecules present in the composition of interest. A “protective immune response” or “therapeutic immune response” refers to an immune response to an antigen derived from an pathogenic antigen (e.g., a tumor antigen from a cancer cell), which in some way prevents, ameliorates, treats (as defined herein) or at least partially arrests disease symptoms, side effects or progression.

As used herein, the term “particle” generally refers to nanoparticles having a diameter between about 1000 nm to less than about 0.1 nm, having a diameter between about 500 and about 10 nm, or more specifically, having a diameter between about 20 nm and about 500 nm. The term also generally refers to microparticles having a diameter between about 0.5 and about 1000 microns, having a diameter between about 1 microns and about 500 microns, or more specifically, having a diameter between about 10 micron and about 100 microns.

As used herein, a “subject” or “patient” means an individual and can include domesticated animals, (e.g., cats, dogs, etc.); livestock (e.g., cattle, horses, pigs, sheep, goats, etc.), laboratory animals (e.g., mouse, rabbit, rat, guinea pig, etc.) and birds. In one aspect, the subject is a mammal such as a primate or a human. In particular, the terms refer to humans diagnosed with cancer.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

### II. POLYMERIC PARTICLES

The methods of the present invention utilize compositions that promote an immune response. In particular embodiments, the composition comprises a particle. In specific embodiments, the particle is a polymeric particle. In one embodiment, the polymeric particle is a microparticle. In another embodiment, the polymeric particle is a nanoparticle. Methods for forming particles, modifying particles (e.g., encapsulating, attaching, or otherwise incorporating on or into particles) are known to those of ordinary skill in the art. See, e.g., U.S. Patent Application Publication No. 2011/0038900, No. 2010/0104503, No. 2009/0269397, and No. 2011/0239789.

Biodegradable or non-biodegradable polymers may be used to form the particles. In particular embodiments, the microparticles are formed of a biodegradable polymer. Non-biodegradable polymers may be used for oral administration. In certain embodiments, synthetic polymers are used, although natural polymers may be used and may have equivalent or even better properties, especially some of the natural biopolymers which degrade by hydrolysis, such as some of the polyhydroxyalkanoates. Representative synthetic polymers include, but are not limited to, poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, celluklose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), cyclodextrins, and copolymers and blends thereof. As used herein, the term “derivatives” includes polymers having substitutions, additions of chemical groups and other modifications routinely made by those skilled in the art. In particular embodiments, PLGA is used as the biodegradable polymer.

Examples of biodegradable polymers useful in the present invention include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), and blends and copolymers thereof.

Natural polymers include, but are not limited to, proteins such as albumin, collagen, gelatin and prolamines, for example, zein, and polysaccharides such as alginate, cellulose derivatives and polyhydroxyalkanoates, for example, polyhydroxybutyrate. The in vivo stability of the particles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

Examples of non-biodegradable polymers include ethylene vinyl acetate, poly(meth)acrylic acid, polyamides, and copolymers and mixtures thereof.

A. Modification of Particles

The external surface of the particles may be modified by conjugating to the surface of the particle a coupling agent or a ligand. In certain embodiments, the ligand is an immunological adjuvant that is encapsulated in or incorporated on or into the particle. Immunological adjuvants include, but are not limited to, Toll-Like Receptor (TLR) ligands, C-Type Lectin Receptor ligands, Nucleotide Oligomerization Domain (NOD)-Like Receptor (NLR) ligands, Retinoic Acid-Inducible Gene-I (RIG)-Like Receptor (RLR) ligands, and Receptor for Advanced Glycation Endproducts (RAGE) ligands.

In particular embodiments, the coupling agent or ligand is present in high density on the surface of the particle. As used herein, the term “high density” refers to particles having a high density of coupling agents or ligands, specifically, in the range of about 1,000 to about 10,000,000, more specifically, about 10,000 to about 1,000,000 coupling agents or ligands per square micron of particle surface area. This can be measured by fluorescence staining of dissolved particles and calibrating this fluorescence to a known amount of free fluorescent molecules in solution.

The particle may be further modified by attachment of one or more different molecules to the ligands or coupling agents, such as targeting molecules, attachment molecules, and/or therapeutic, nutritional, diagnostic or prophylactic agents. A targeting molecule is a substance that will direct the particle to a receptor site on a selected cell or tissue type, can serve as an attachment molecule, or serve to couple or attach another molecule. As used herein, “direct” refers to causing a molecule to preferentially attach to a selected cell or tissue type. This can be used to direct cellular materials, molecules, or drugs, as discussed below.

The particles are designed to release encapsulated or attached molecules over a period of days to weeks. Factors that affect the duration of release include pH of the surrounding medium (higher rate of release at pH 5 and below due to acid catalyzed hydrolysis of PLGA) and polymer composition. By varying the polymer composition of the particle and morphology, one can effectively tune in a variety of controlled release characteristics allowing for moderate constant doses over prolonged periods of time. There have been a variety of materials used to engineer solid particles with and without surface functionality. See Brigger et al., 54 ADV. DRUG DELIV. REV. 631-51 (2002). Perhaps the most widely used materials are the aliphatic polyesters, specifically, the hydrophobic poly (lactic acid) (PLA), more hydrophilic poly (glycolic acid) PGA and their copolymers, poly (lactide-co-glycolide) (PLGA). The degradation rate of these polymers, and often the corresponding drug release rate, can vary from days (PGA) to months (PLA) and is easily manipulated by varying the ratio of PLA to PGA. The physiologic compatibility of PLGA and its hompolymers PGA and PLA have been established for safe use in humans. These materials have a history of over 30 years in various human clinical applications including drug delivery systems. Furthermore, PLGA particles can be formulated in a variety of ways that improve drug pharmacokinetics and biodistribution to target tissue by either passive or active targeting. In particular embodiments, the polymers exhibit degradation kinetics lasting between about 1 and about 30 days.

B. Formation of Particles

Particles can be fabricated from different polymers using different methods. In the solvent evaporation method, the polymer is dissolved in a volatile organic solvent, such as methylene chloride. The therapeutic agent (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid particles. The resulting particles are washed with water and dried overnight in a lyophilizer. Particles with different sizes (about 0.5 to about 1000 microns) and morphologies can be obtained by this method. This method is useful for relatively stable polymers like polyesters and polystyrene.

The hot melt encapsulation method and the solvent removal method may be used for labile polymers, such as polyanhydrides, which can degrade during the fabrication process due to the presence of water. In the hot melt encapsulation method, the polymer is first melted and then mixed with the solid particles. The mixture is suspended in a non-miscible solvent (like silicon oil), and, with continuous stirring, heated to 5° C. above the melting point of the polymer. Once the emulsion is stabilized, it is cooled until the polymer particles solidify. The resulting particles are washed by decantation with petroleum ether to give a free-flowing powder. Particles with sizes between about 0.5 to about 1000 microns can be obtained with this method. The external surfaces of spheres prepared with this technique are usually smooth and dense. This procedure is used to prepare particles made of polyesters and polyanhydrides. However, this method is generally limited to polymers with molecular weights between about 1.000 to about 50,000.

The solvent removal technique is primarily designed for polyanhydrides. In this method, the therapeutic agent is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. Particles that range between about 1 to about 300 microns can be obtained by this procedure. The external morphology of spheres produced with this technique is highly dependent on the type of polymer used.

Spray-drying is another method useful for forming particles of the present invention. In this method, the polymer is dissolved in organic solvent. A known amount of the therapeutic agent is suspended (insoluble drugs) or co-dissolved (soluble drugs) in the polymer solution. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting=15, pump setting=10 mL/minute, spray flow=600 Nl/hr. and nozzle diameter=0.5 mm. Particles ranging between about 1 to about 10 microns can be obtained with a morphology which depends on the type of polymer used.

Particles made of gel-type polymers, such as alginate, are produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The particles are left to incubate in the bath for twenty to thirty minutes in order to allow sufficient time for gelation to occur. Particle size is controlled by using various size extruders or varying either the nitrogen gas or polymer solution flow rates. Chitosan particles can be prepared by dissolving the polymer in acidic solution and crosslinking it with tripolyphosphate. Carboxymethyl cellulose (CMC) particles can be prepared by dissolving the polymer in acid solution and precipitating the particle with lead ions. In the case of negatively charged polymers (e.g., alginate, CMC), positively charged ligands (e.g., polylysine, polyethyleneimine) of different molecular weights can be ionically attached.

### II. MOLECULES ASSOCIATED WITH THE PARTICLES

In the present invention, a composition that promotes an immune response is administered to a patient. The composition can comprise a particle described herein. In certain embodiments, the external surface of the particle is coated with a coupling agent and/or a ligand. The ligand can be an immunological adjuvant. The adjuvant can be entrapped within the particle, associated with the surface of the particle (e.g., adsorbed or conjugated (directly or indirectly) to the particle surface), and/or otherwise associated with the particle to varying degrees (e.g., admixed with particles in a liquid suspension, admixed with the particles in a solid composition, for instance, co-lyophilized with the particles, etc.), among other possibilities. Examples of immunological adjuvants that can be associated with the particles include, but are not limited to, TLR ligands, C-Type Lectin Receptor ligands, NLR ligands, RLR ligands, and RAGE ligands. TLR ligands can include lipopolysaccharide (LPS) and derivatives thereof, as well as lipid A and derivatives there of including, but not limited to, monophosphoryl lipid A (MPL), glycopyranosyl lipid A, PET-lipid A, and 3-O-desacyl-4′-monophosphoryl lipid A. In a specific embodiment, the immunological adjuvant is MPL. In another embodiment, the immunological adjuvant is LPS. TLR ligands can also include, but are not limited to, TLR3 ligands (e.g., polyinosinic-polycytidylic acid (poly(I:C)). TLR7 ligands (e.g. imiquimod and resiquimod), and TLR9 ligands.

It is within the scope of the present invention to utilize particles in which one or more coating agents and/or ligands are encapsulated in or incorporated on or into the particle. For example, the compositions comprising a polymeric particle may further comprise one or more immunological adjuvants encapsulated in or incorporated on or into the particle. The compositions can further comprise one or more therapeutic agents encapsulated in or incorporated on or into the particle. In a specific embodiment, a TLR4 ligand and a TLR7 ligand can be encapsulated in or incorporated on or into a particle. In a more specific embodiment, MPL (TLR4 ligand) and R837 (TLR7 ligand) can be encapsulated in or incorporated on or into a particle. Such a particle can also comprise a therapeutic agent, such as an antigen. See Kasturi et al., 470 NATURE 543-50 (2011).

In further embodiments, other molecules can be encapsulated in or incorporated on or into the particles. In certain embodiments, a therapeutic agent is encapsulated in or incorporated on or into the particles. Therapeutic agents can include, but are not limited to, tumor antigens. CD4⁺ T-cell epitopes, cytokines, chemotherapeutic agents, radionuclides, small molecule signal transduction inhibitors, photothermal antennas, small interfering RNAs, monoclonal antibodies, and immunologic danger signaling molecules.

In particular embodiments, one or more antigens may optionally be provided in the compositions of the invention. Antigens may be entrapped within the nanoparticles, associated with the surfaces of the nanoparticles (e.g., adsorbed or conjugated to the surfaces of the nanoparticles) and/or otherwise associated with the nanoparticles to varying degrees (e.g., admixed with the nanoparticles in a liquid suspension, admixed with the nanoparticles in a solid composition, for instance, co-lyophilized with the nanoparticles), among other possibilities.

Each antigen may be provided in an effective amount (e.g., an amount effective for use in therapeutic, prophylactic, or diagnostic methods in accordance with the invention). Antigens useful in the present invention include, but are not limited to, bacterial antigens, viral antigens, fungal antigens, and tumor or cancer antigens.

The compositions of the invention can include one or more tumor or cancer antigens. Tumor antigens can be, for example, peptide-containing tumor antigens, such as a polypeptide tumor antigen or glycoprotein tumor antigens. A tumor antigen can also be, for example, a saccharide-containing tumor antigen, such as a glycolipid tumor antigen or a ganglioside tumor antigen. A tumor antigen can further be, for example, a polynucleotide-containing tumor antigen that expresses a polypeptide-containing tumor antigen, for instance, an RNA vector construct or a DNA vector construct, such as plasmid DNA.

Tumor antigens include, but are not limited to, (a) polypeptide-containing tumor antigens, including polypeptides (which can range, for example, from about 8 to about 20 amino acids in length, although lengths outside this range are also common), lipopolypeptides and glycoproteins, (b) saccharide-containing tumor antigens, including poly-saccharides, mucins, gangliosides, glycolipids and glycoproteins, and (c) polynucleotides that express antigenic polypeptides.

Moreover, tumor antigens can be (a) full length molecules associated with cancer cells, (b) homologs and modified forms of the same, including molecules with deleted, added and/or substituted portions, (c) fragments of the same, and (d) extracts or lysates of tumor cells. Tumor antigens can also be provided in recombinant form. Tumor antigens include, for example, class I-restricted antigens recognized by CD8⁺ lymphocytes or class II-restricted antigens recognized by CD4⁺ lymphocytes.

Numerous tumor antigens are known in the art, including: (a) cancer-testis antigens such as NY-ESO-1, SSX2. SCP1 as well as RAGE, BAGE, GAGE and MAGE family polypeptides, for example, GAGE-1, GAGE-2, MAGE-1. MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors), (b) mutated antigens, for example, p53 (associated with various solid tumors, e.g., colorectal, lung, head and neck cancer), p21/Ras (associated with, e.g. melanoma, pancreatic cancer and colorectal cancer), CDK4 (associated with, e.g. melanoma), MUM1 (associated with, e.g., melanoma), caspase-8 (associated with, e.g., head and neck cancer), CIA 0205 (associated with, e.g., bladder cancer), HLA-A2-R 1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT. (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g. renal cancer), aldolase A (associated with, e.g., lung cancer), PRAME (associated with, e.g. melanoma). HER-2/neu (associated with, e.g., breast, colon, lung and ovarian cancer), alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), and carcinoembryonic antigen (associated with, e.g., breast cancer, lung cancer, and cancers of the gastrointestinal tract such as colorectal cancer), (d) shared antigens, for example, melanoma-melanocyte differentiation antigens such as MART-1/Melan A, gp100, MC1R, melanocyte-stimulating hormone receptor, tyrosinase, tyrosinase related protein-1/TRP1 and tyrosinase related protein-2/TRP2 (associated with, e.g., melanoma), (e) prostate associated antigens such as PAP, PSA, PSMA, PSH-P1, PSM-P1, PSM-P2, associated with e.g. prostate cancer, (f) immunoglobulin idiotypes (associated with myeloma and B cell lymphomas, for example), and (g) other tumor antigens, such as polypeptide- and saccharide-containing antigens including (i) glycoproteins such as sialyl Tn and sialyl Le^{x} (associated with, e.g., breast and colorectal cancer) as well as various mucins; glycoproteins may be coupled to a carrier protein (e.g., MUC-1 may be coupled to KLH); (ii) lipopolypeptides (e.g., MUC-1 linked to a lipid moiety); (iii) polysaccharides (e.g., Globo H synthetic hexasaccharide), which may be coupled to a carrier proteins (e.g., to KLH), (iv) gangliosides such as GM2, GM12, GD2, GD3 (associated with, e.g., brain, lung cancer, melanoma), which also may be coupled to carrier proteins (e.g., KLH).

Other tumor antigens include p15. Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, p185erbB2, p180erbB-3, c-met, mn-23H1, TAG-72-4, CA 19-9, CA 72-4, CAM 17.1, NuMa, K-ras, p16, TAGE, PSCA, (CT7, 43-9F, 5T4, 791 Tgp72, beta-HCG, BCA225, BTAA, CA 125, CA 15-3 (CA 27.29\BCAA). CA 195, CA 242, CA-50, CAM43, CD68\KP1, CO-029, FGF-5, Ga733 (EpCAM), 1HTgp-175, M344, MA-50, MG7-Ag, MOV18, NB/70K, NY-CO-1. RCAS1, SDCCAG16, TA-90 (Mac-2 binding protein\cyclophilin C-associated protein), TAAL6, TAG72, TLP, TPS, and the like.

Polynucleotide-containing antigens in accordance with the present invention typically comprise polynucleotides that encode polypeptide cancer antigens such as those listed above. Particular polynucleotide-containing antigens include DNA or RNA vector constructs, such as plasmid vectors (e.g., pCMV), which are capable of expressing polypeptide cancer antigens in vivo.

Tumor antigens may be derived, for example, from mutated or altered cellular components. After alteration, the cellular components no longer perform their regulatory functions, and hence the cell may experience uncontrolled growth. Representative examples of altered cellular components include ras, p53, Rb, altered protein encoded by the Wilms' tumor gene, ubiquitin, mucin, protein encoded by the DCC, APC, and MCC genes, as well as receptors or receptor-like structures such as neu, thyroid hormone receptor, platelet derived growth factor (PDGF) receptor, insulin receptor, epidermal growth factor (EGF) receptor, and the colony stimulating factor (CSF) receptor. These as well as other cellular components are described for example in U.S. Pat. No. 5,693,522 and references cited therein.

Bacterial and viral antigens may be used in conjunction with the compositions of the present invention for the treatment of cancer. In particular, carrier proteins, such as CRM₁₉₇, tetanus toxoid, or Salmonella typhimurium antigen may be used in conjunction/conjugation with compounds of the present invention for treatment of cancer. The cancer antigen combination therapies can show increased efficacy and bioavailability as compared with existing therapies.

### III. AGENTS THAT MITIGATE THE SUPPRESSION OF ANTI-TUMOR IMMUNITY

The methods of the present invention also comprise the administration of agents that mitigate the suppression of anti-tumor immunity. In certain embodiments, the agents destroy or otherwise inhibit regulatory T cells. In other embodiments, the agents alter the composition of antigen-presenting cells in the tumor microenvironment, e.g. killing myeloid-derived suppressor cells. Example of agents that mitigate suppression of anti-tumor immunity include, but are not limited to, alkylating agents, steroids, nucleotide inhibitory drugs, chemotherapeutics, monoclonal antibodies, toxins, and inflammatory reducing agents. More specific examples include, but are not limited to, cyclophosphamide, 5-fluorouracil, gemcitabine, doxorubicin, denileukin, diftitox, bevacizumab, and docetaxel. In a specific embodiment, the agent is cyclophosphamide (also referred to as Cy or Cytoxan). Such agents can be administered prior to or after the particle composition.

### IV. TISSUE ABLATION

According to the present invention, various methods for physical destruction of cancer cells can be used in conjunction with the administration of agents that mitigate suppression of anti-tumor immunity and/or compositions that promote or induce an immune response. As used herein, the terms “ablate,” “ablation,” “ablating” and derivatives thereof refer to the substantial alteration of tissue, specifically, cancerous tissue or cells or tumors. The term also applies to the alteration of any hyperplastic growth or pre-malignant lesion, such as a dysplastic nevus, colonic polyp, fibroadenoma, uterine fibroid, molluscum contagiosum, cervical papilloma, or skin wart. In certain embodiments, the term “ablation” refers to the physical destruction of the target cell, e.g., cancer cells. More specifically, the term can mean the direct necrosis of tissue. In particular embodiments, any method of ablation that leads to cells/tissues releasing tumor antigens and/or immunologic danger signals such as High Mobility Group Box 1 protein (HMGB1), adenosine triphosphate (ATP)), heat shock proteins (HSPs), S100 proteins, SAP130, RNA, double-stranded genomic DNA, hyaluronan, biglycan, versican, heparan sulphate, mitochondrial formyl peptides, mitochondrial DNA, calcium pyrophosphate dehydrate crystals, beta-amyloid, cholesterol crystals, interleukin (IL)-1 alpha, IL-33, or crystals of uric acid or monosodium urate, which promote anti-tumor immunity. For example, cryoablation, i.e., freezing of cells, disrupts cell membranes and releases intact tumor antigens, which are captured by antigen-presenting cells for presentation to antitumor lymphocytes in tumor-draining lymph nodes. See den Brok et al., 95 BR. J. CANCER 896-905 (2006). Certain embodiments involving particles coated with immunological adjuvants (e.g., a MPL-coated particles) can result in the decrease the toxicity of subsequent tumor cryoablation because MPL “turns off” the inflammatory signaling induced by the release of endogenous danger signals.

Accordingly, methods for ablating tissue can include, but are not limited to, cryoablation, thermal ablation, photoacoustic ablation, radiotherapy, chemotherapy, radiofrequency ablation, electroporation, alcohol ablation, high-intensity focused ultrasound, photodynamic therapy, monoclonal antibodies, immunotoxins, and the like.

Tumor ablation technology for medical treatment is known in the art and includes such treatment modalities as radiofrequency (RF), focused ultrasound, such as high intensity ultrasound beams, microwave, laser, thermal electric heating, traditional heating methods with electrodes using direct current (DC) or alternating current (AC), and application of heated fluids and cold therapies (such as cryosurgery, also known as cryotherapy or cryoablation. For information on cryoablation, see CSA Medical, Inc. (Baltimore, Md.) (U.S. Pat. No. 7,255,693; No. 6,027,499; and No. 6,383,181); Endocare, Inc./HealthTronics, Inc. (Austin, Tex.) (U.S. Pat. No. 7,921,657; No. 7,621,889; No. 6,972,014; No. 6,936,045; No. 6,544,176; and 6,251,105; U.S. Patent Application Publication No. 20110040297; No. 20110009854; No. 20100180607; and No. 20020087152); and Galil Medical (Arden Hills, Minn.) (U.S. Pat. No. 7,942,870; No. 7,850,682: No. 7,846,154; No. 7,625,369; No. 7,604,605; No. 7,479,139; No. 6,905,492; and No. 6,875,209; U.S. Patent Application Publication No. 20110009748; No. 20100019918; No. 20100168567; No. 20100152722; No. 20090306639; No. 20090306638; and No. 20080045934).

Irreversible electroporation (IRE) is a nonthermal ablation technique that induces cell necrosis without raising the temperature of the ablation zone. More specifically IRE is a technology where electrical pulses in the range of nanoseconds to milliseconds are applied to tissue to produce cellular necrosis and irreversible cell membrane permeabilization. More precisely, IRE treatment acts by creating defects in the cell membrane that are nanoscale in size and that lead to a disruption of homeostasis while sparing connective and scaffolding structure and tissue. See U.S. Patent Application Publication No. 2007/0043345 and No. 2006/0293731, as well as International Patent Application Publication Number WO2005/06284A2.

### V. COMPOSITION FORMULATION AND ADMINISTRATION

Accordingly, particular embodiments of the methods of the present invention relate to the administration of effective amounts of compositions including, agents that mitigate suppression of anti-tumor immunity (e.g., cyclophosphamide, denileukin, etc.), and particles described herein (e.g., nanoparticles coated with immunological adjuvants and/or encapsulating a therapeutic agent(s)). As used herein, the term “effective,” means adequate to accomplish a desired, expected, or intended result. More particularly, an “effective amount” or a “therapeutically effective amount” is used interchangeably and refers to an amount of a composition of the present invention (e.g., cyclophosphamide and/or a particle), either alone or in combination with another therapeutic agent, necessary to provide the desired therapeutic effect, e.g., an amount that is effective to prevent, alleviate, treat or ameliorate symptoms of disease or prolong the survival of the subject being treated. As would be appreciated by one of ordinary skill in the art, the exact amount required will vary from subject to subject, depending on age, general condition of the subject, the severity of the condition being treated, the particular compound and/or composition administered, and the like. An appropriate “therapeutically effective amount” in any individual case can be determined by one of ordinary skill in the art by reference to the pertinent texts and literature and/or by using routine experimentation. It is understood that reference to a pharmaceutical composition, its formulation, administration, and the like, can refer to, depending on the context, one or more of agents that mitigate suppression of anti-tumor immunity (e.g., cyclophosphamide, denileukin, etc.), and particles described herein (e.g., nanoparticles coated with immunological adjuvants and/or encapsulating a therapeutic agents).

The compositions of the present invention are in biologically compatible form suitable for administration in vive for subjects. The pharmaceutical compositions further comprise a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the cit-PAD4 polypeptide is administered. Such pharmaceutical carriers can be sterile liquids, such as water and oils, including those of petroleum, animal, vegetable or synthetic origin, including but not limited to peanut oil, soybean oil, mineral oil, sesame oil and the like. Water may be a carrier when the pharmaceutical composition is administered orally. Saline and aqueous dextrose may be carriers when the pharmaceutical composition is administered intravenously. Saline solutions and aqueous dextrose and glycerol solutions may be employed as liquid carriers for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition may also contain minor amounts of wetting or emulsifying agents, or pH buffering agents.

The pharmaceutical compositions of the present invention can take the form of solutions, suspensions, emulsions, sustained-release formulations and the like. In a specific embodiment, a pharmaceutical composition comprises an effective amount of a particle of the present invention together with a suitable amount of a pharmaceutically acceptable carrier so as to provide the form for proper administration to the patient. The formulation should suit the mode of administration.

The pharmaceutical compositions of the present invention may be administered by any particular route of administration including, but not limited to oral, parenteral, subcutaneous, intramuscular, intravenous, intraperitoneal, intrapleural, intraprostatic, intrapulmonary, sublingual or intranasal means. Most suitable routes are by intravenous, intramuscular or subcutaneous injection. In particular embodiments, the compositions are administered at or near the target area, e.g., intratumoral injection.

In general, the pharmaceutical compositions may be used alone or in concert with other therapeutic agents at appropriate dosages defined by routine testing in order to obtain optimal efficacy while minimizing any potential toxicity. The dosage regimen utilizing a pharmaceutical composition of the present invention may be selected in accordance with a variety of factors including type, species, age, weight, sex, medical condition of the patient; the severity of the condition to be treated; the route of administration; the renal and hepatic function of the patient; and the particular pharmaceutical composition employed. A physician of ordinary skill can readily determine and prescribe the effective amount of the pharmaceutical composition (and potentially other agents including therapeutic agents) required to prevent, counter, or arrest the progress of the condition.

Optimal precision in achieving concentrations of the therapeutic regimen within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. The dosages of a pharmaceutical composition disclosed herein may be adjusted when combined to achieve desired effects. On the other hand, dosages of the pharmaceutical composition and various therapeutic agents may be independently optimized and combined to achieve a synergistic result wherein the pathology is reduced more than it would be if either was used alone.

In particular, toxicity and therapeutic efficacy of the pharmaceutical composition may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between to toxic and therapeutic effect is the therapeutic index and it may be expressed as the ratio LD₅₀/ED₅₀. Pharmaceutical compositions exhibiting large therapeutic indices are preferred except when cytotoxicity of the composition is the activity or therapeutic outcome that is desired. Although pharmaceutical compositions that exhibit toxic side effects may be used, a delivery system can target such compositions to the site of affected tissue in order to minimize potential damage to uninfected cells and, thereby, reduce side effects. Generally, the pharmaceutical compositions of the present invention may be administered in a manner that maximizes efficacy and minimizes toxicity.

Data obtained from cell culture assays and animal studies may be used in formulating a range of dosages for use in humans. The dosages of such compositions lie preferably within a range of circulating concentrations that include the ED₅₀ with little or no toxicity. The dosage may vary within this range depending upon the dosage form employed and the route of administration utilized. For any composition used in the methods of the invention, the therapeutically effective dose may be estimated initially from cell culture assays. A dose may be formulated in animal models to achieve a circulating plasma concentration range that includes the IC₅₀ (the concentration of the test composition that achieves a half-maximal inhibition of symptoms) as determined in cell culture. Such information may be used to accurately determine useful doses in humans. Levels in plasma may be measured, for example, by high performance liquid chromatography.

Moreover, the dosage administration of the compositions of the present invention may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile. See WO 00/67776, which is entirely expressly incorporated herein by reference.

More specifically, the pharmaceutical compositions may be administered in a single daily dose, or the total daily dosage may be administered in divided doses of two, three, or four times daily. In the case of oral administration, the daily dosage of the compositions may be varied over a wide range from about 0.1 ng to about 1,000 mg per patient, per day. The range may more particularly be from about 0.001 ng/kg to 10 mg/kg of body weight per day, about 0.1-100 μg, about 1.0-50 μg or about 1.0-20 mg per day for adults (at about 60 kg).

The daily dosage of the pharmaceutical compositions may be varied over a wide range from about 0.1 ng to about 1000 mg per adult human per day. For oral administration, the compositions may be provided in the form of tablets containing from about 0.1 ng to about 1000 mg of the composition or 0.1, 0.2, 0.5, 1.0, 2.0, 5.0, 10.0, 15.0, 100, 150, 200, 250, 300, 350, 400, 450, 500, 550, 600, 650, 700, 800, 900, or 1000 milligrams of the composition for the symptomatic adjustment of the dosage to the patient to be treated. An effective amount of the pharmaceutical composition is ordinarily supplied at a dosage level of from about 0.1 ng/kg to about 20 mg/kg of body weight per day. In one embodiment, the range is from about 0.2 ng/kg to about 10 mg/kg of body weight per day. In another embodiment, the range is from about 0.5 ng/kg to about 10 mg/kg of body weight per day.

The pharmaceutical compositions may be administered on a regimen of about 1 to about 10 times per day.

In the case of injections, it is usually convenient to give by an intravenous route in an amount of about 0.0001 μg-30 mg, about 0.01 μg-20 mg or about 0.01-10 mg per day to adults (at about 60 kg). In the case of other animals, the dose calculated for 60 kg may be administered as well.

Doses of a pharmaceutical composition of the present invention can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1.0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof. In particular embodiments, a particle of the present invention may be administered in a dose of about 1 μg to about 1 g. In other embodiments, cyclophosphamide can be administered in a dose of about 50 mg/day orally to about 50 mg/kg/day intravenously. In one embodiment, about 50 mg of cyclophosphamide is administered orally twice a day for at least about two weeks prior to ablation. In an alternative embodiment, about 50 mg/kg of cyclophosphamide is administered intravenously on the day before ablation.

As a non-limiting example, treatment of subjects can be provided as a one-time or periodic dosage of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Specifically, the pharmaceutical compositions of the present invention may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

More specifically, the pharmaceutical compositions may be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

Alternatively, the pharmaceutical compositions may be administered about once every day, about once every 2 days, about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days.

The pharmaceutical compositions of the present invention may alternatively be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks.

Alternatively, the pharmaceutical compositions of the present invention may be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

Alternatively, the pharmaceutical compositions may be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

Alternatively the pharmaceutical compositions may be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

It would be readily apparent to one of ordinary skill in the art that the pharmaceutical compositions of the present invention can be combined with one or more therapeutic agents. In particular, the compositions of the present invention and other therapeutic agents can be administered simultaneously or sequentially by the same or different routes of administration. The determination of the identity and amount of therapeutic agent(s) for use in the methods of the present invention can be readily made by ordinarily skilled medical practitioners using standard techniques known in the art. In specific embodiments, the particles of the present invention can be administered in combination with an effective amount of a second therapeutic agent that treats cancer.

In another aspect, the particles of the present invention may be combined with other therapeutic agents including, but not limited to, immunomodulatory agents; anti-inflammatory agents (e.g., adrenocorticoids, corticosteroids (e.g., beclomethasone, budesonide, flunisolide, fluticasone, triamcinolone, methlyprednisolone, prednisolone, prednisone, hydrocortisone), glucocorticoids, steroids, and non-steriodal anti-inflammatory drugs (e.g., aspirin, ibuprofein, diclofenac, and COX-2 inhibitors) and leukotreine antagonists (e.g., montelukast, methyl xanthines, zafirlukast, and zileuton); beta2-agonists (e.g., albuterol, biterol, fenoterol, isoetharie, metaproterenol, pirbuterol, salbutamol, terbutalin formoterol, salmeterol, and salbutamol terbutaline); anticholinergic agents (e.g., ipratropium bromide and oxitropium bromide), sulphasalazine, penicillamine, dapsone, antihistamines, anti-malarial agents (e.g., hydroxychloroquine); anti-viral agents; and antibiotics (e.g., dactinomycin (formerly actinomycin), bleomycin, erythomycin, penicillin, mithramycin, and anthramycin (AMC)).

In various embodiments, the compositions of the present invention in combination with a second therapeutic agent may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In particular embodiments, two or more therapies are administered within the same patent visit.

In certain embodiments, the particles of the present invention and one or more other therapies are cyclically administered. Cycling therapy involves the administration of a first therapy (e.g., the particles of the present invention) for a period of time, followed by the administration of a second therapy (e.g. another therapeutic agent) for a period of time, optionally, followed by the administration of a third therapy for a period of time and so forth, and repeating this sequential administration, e.g. the cycle, in order to reduce the development of resistance to one of the therapies, to avoid or reduce the side effects of one of the therapies, and/or to improve the efficacy of the therapies. In certain embodiments, the administration of the combination therapy of the present invention may be repeated and the administrations may be separated by at least 1 day, 2 days, 3 days, 5 days, 10 days, 15 days, 30 days, 45 days, 2 months, 75 days, 3 months, or at least 6 months.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Nanoparticle Construction.

50:50 PLGA with an inherent viscosity of 0.59 dL/g, is purchased from Lactel Polymers. Inc. (Pelham, Ala., USA). Polyvinyl alcohol (PVA) (M_{w} average 30-70 kD) and LPS (Escherichia coli strain 0111:B4), are obtained from Sigma. Aldrich. Chromatography grade methylene chloride is supplied by Fisher Scientific.

Preparation of LPS-Modified-Biodegradable Nanoparticles.

A modified water-in-oil-in-water (W/O/W) emulsion method is used for preparation of LPS-modified PLGA particles. The first emulsion can be used to encapsulate molecules such as tumor antigens, antigens for CD4⁺ T cells, cytokines, danger molecules, drugs, radionuclides, or small molecules into the core of the nanoparticle. To incorporate an antigen into the nanoparticle in the first emulsion (W/O), super-concentrated antigen (20-100 mg/ml) in phosphate-buffered saline (PBS) is added drop-wise to a vortexing PLGA solution (2 ml) dissolved in methylene chloride. Polymer and encapsulant are then added drop-wise to 5% PVA in the second emulsion (W/O/W). After each emulsion, the samples are sonicated for 30 seconds on ice using a Tekmar Sonic Distributor fitted with a model CV26 sonicator—amplitude set at 38%. The second emulsion is rapidly added to 0.3% PVA. This external phase is stirred vigorously for 3 hours at constant room temperature to evaporate methylene chloride. LPS-modified particles are prepared with LPS (20 mg/ml in de-ionized (DI) water) added to the second emulsion containing 5% PVA. Particles are collected at 12,000 rpm for 15 min. and washed with DI water three times. The particles are freeze dried and stored at −20° C. for later use. Nanoparticles are stable at −20° C. for years.

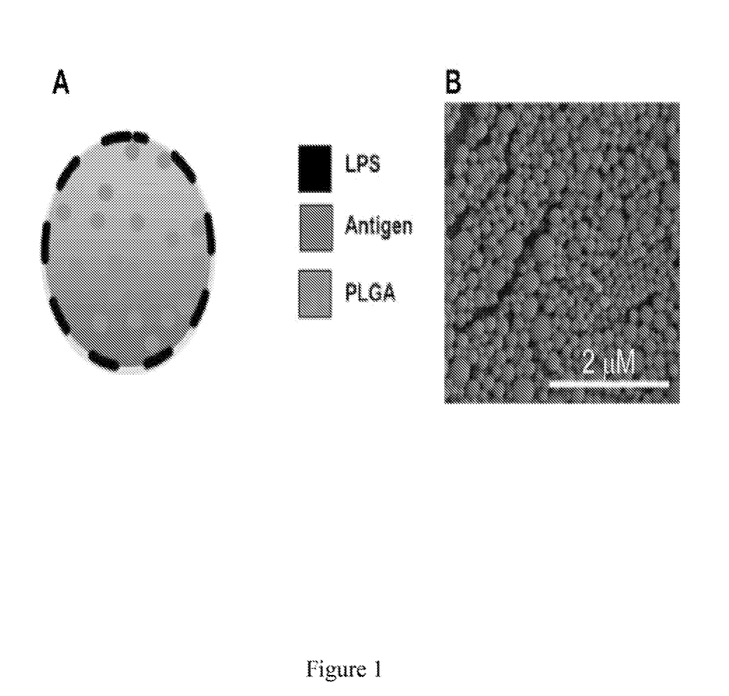

A schematic diagram of LPS-modified, antigen-encapsulated nanoparticles is shown in FIG. 1A. FIG. 1B shows a scanning electron micrograph of nanoparticles prepared as above.

Intratumoral Administration of Nanoparticles.

The nanoparticles are taken from the freezer, weighed, and then suspended in 1×PBS to a concentration of 1 mg/ml. The particles are suspended with a pipette for about one minute. Once mixed with PBS, nanoparticles that are kept at 4° C. may be used for up to 12 hours. The particles in PBS should not be used if kept for more than 12 hours. The particles are lightly sonicated with a bath sonicator, or vortexed to make an even suspension. The particles should not sit idly in the syringe as they will settle and then clog the syringe. Ideally, the injection should be made within 10-30 seconds after drawing the well-mixed nanoparticle suspension into the syringe. For mice with a tumor of 5-8 mm diameter, 100 mg of particles is injected directly into the tumor in a volume of 0.1 ml.

### Example 1

### Immunotherapy of Metastatic Cancer in Mice

It was hypothesized that tumor cryoablation releases tumor antigens and immunologic danger signals such as HMGB1 and ATP, which promote anti-tumor immunity. It was further hypothesized that the particles of the present invention could enhance the immunogenicity of cryoimmunotherapy at the time of tumor antigen presentation.

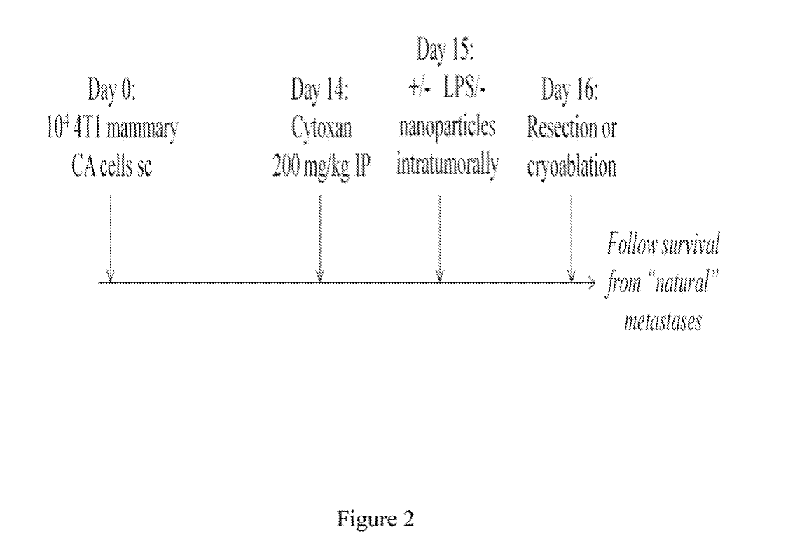

Groups of 10 BALB/c mice each received a subcutaneous inoculation of 10⁴ cells of the highly metastatic, syngeneic mammary cancer cell line on the flank. Beginning on day 14, when 3-5 mm tumor nodules are present at the site of injection, mice were treated with permutations of cyclophosphamide 200 mg/kg intraperitoneally on day 14, LPS-modified nanoparticles (100 μg/mouse) intratumorally on day 15, and either resection or cryoablation of the tumor on day 16. See FIG. 2.

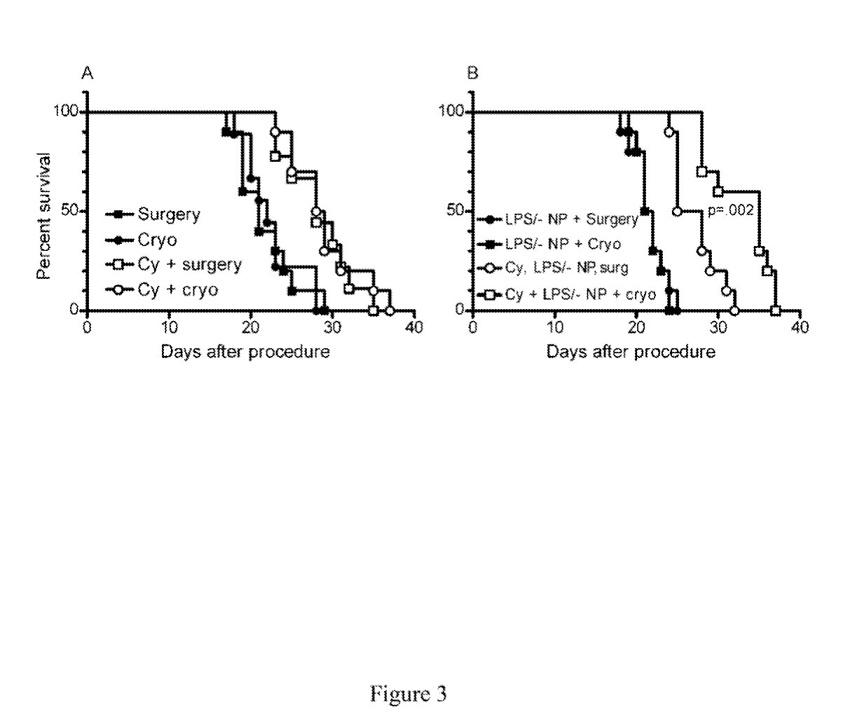

FIG. 3A shows that cyclophosphamide (Cy or Cytoxan) does not unmask a systemic anti-tumor effect of cryoablation, as animals treated with Cy plus cryoablation did not survive significantly longer than animals treated with Cy plus surgical resection of the subcutaneous nodule. In contrast, tumor-bearing mice treated with Cy, nanoparticles, and cryoablation survived significantly longer than tumor-bearing mice treated with Cy, nanoparticles, and surgical resection (panel B; p=0.002). This result raises the possibility that cryoablation of the 4T1 tumor liberates tumor antigens but does not liberate sufficient danger signals to stimulate anti-tumor immunity, but this deficiency can be overcome by injecting nanoparticles prior to cryoablation. In summary, these results suggest that nanoparticles provide an adjuvant effect to stimulate immunity against tumor antigens liberated by local tumor cryoablation, resulting in prolonged survival of animals with metastatic cancer.

### Example 2 (Prophetic)

### Immunotherapy of Prostate Cancer in Patients

A proprietary tumor antigen, such as Dendreon's prostatic acid phosphatase-GM-CSF fusion protein, is encapsulated into LPS-modified nanoparticles. The tumor antigen-containing nanoparticles are injected into the prostate tumor bed just prior to local tumor cryoablation. This treatment can be applied to a patient with newly diagnosed prostate cancer, prostate cancer recurring locally after definitive radiation therapy, or metastatic prostate cancer. The immune response to cryoimmunotherapy can be boosted with intermittent subcutaneous injections of Sipuleucel-T (Provenge®) (Dendreon Corp., Seattle, Wash.)). The patient may be treated with cyclophosphamide 50 mg daily by mouth for two weeks to deplete regulatory T cells prior to cryoablation.

### Example 3 (Prophetic)

### Immunotherapy of Breast Cancer in Patients

The approach described in Example 2 can be applied to patients with breast cancer by encapsulating Her2/neu epitopes into LPS-modified nanoparticles, injection of the nanoparticles into a breast cancer prior to cryoablation, and boosting with Lapuleucel-T (Dendreon Corp., Seattle, Wash.).

### Example 4 (Prophetic)

### Immunotherapy of Kidney Cancer in Patients

The approach described in Example 2 can be applied to patients with kidney cancer by incorporating carbonic anhydrase-9 into LPS-modified nanoparticles for cryoimmunotherapy of renal cell carcinoma.

### Example 5

### Immunotherapy of Cancer in Patients

A pool of overlapping pentadecapeptides from the immunodominant pp65 protein of human cytomegalovirus is encapsulated into LPS-modified nanoparticles and injected just prior to cryoablation into the tumor of a CMV-seropositive individual. This strategy will attract CMV-specific CD4⁺ T cells to the site of the ablated tumor or to the tumor-draining lymph node, where the cells can provide help for the sustained activation of tumor-specific CD8⁺ T cells.

Alternatively, a cancer patient can be vaccinated with an antigen containing CD4⁺ T cell epitopes. More than two weeks later (to allow the generation of antigen-specific memory CD4⁺ T cells) and just prior to cryoablation, the patient receives an intratumoral injection of LPS-modified nanoparticles containing the antigen or CD4⁺ T cell epitopes in the antigen. This strategy will also attract memory CD4⁺ T cell help to the sites where CD8⁺ T cells are encountering tumor antigens.

### REFERENCES

1. Demento et al., 27 VACCINE 3013-21 (2009).
2. Levy et al., 330 J. PHARMACOL. EXP. THER. 596-601 (2009).
3. Glasgow 293 AM. J. PHYSIOL. LUNG CELL. MOL. PHYSIOL. L491-L496 (2007).

1. A method for treating cancer in a patient comprising the steps of:
a. administering at or near the cancer site an effective amount of a composition that promotes a therapeutic immune response to the cancer; and
b. ablating the cancer.

2. The method of claim 1, further comprising administering an effective amount of an agent that mitigates suppression of anti-tumor immunity to the patient prior to or after administering the composition.

3. The method of claim 2, wherein the agent is selected from the group consisting of alkylating agents, steroids, nucleotide inhibitory drugs, chemotherapeutics, monoclonal antibodies, toxins, and inflammatory reducing agents.

4. The method of claim 2, wherein the agent is selected from the group consisting of cyclophosphamide, 5-fluorouracil, gemcitabine, doxorubicin, denileukin, diftitox, bevacizumab, and docetaxel.

5. The method of claim 1, wherein the composition comprises (a) a polymeric particle; and (b) optionally one or more therapeutic agents encapsulated in or incorporated on or into the polymeric particle.

6. The method of claim 5, wherein the polymeric particle comprises poly lactide (PLA), polyglycolide (PGA), poly(lactic-co-glycolic acid) (PLGA) or co-polymers thereof.

7. The method of claim 5, wherein the polymeric particle is PLGA.

8. The method of claim 5, wherein the composition further comprises one or more immunological adjuvants encapsulated in or incorporated on or into the polymeric particle.

9. The method of claim 8, wherein the immunological adjuvant is a Toll-Like Receptor (TLR) Ligand.

10. The method of claim 8, wherein the immunological adjuvant is a C-Type Lectin Receptor Ligand.

11. The method of claim 8, wherein the immunological adjuvant is a Nucleotide Oligomerization Domain (NOD)-Like Receptor Ligand.

12. The method of claim 8, wherein the immunological adjuvant is a Retinoic Acid-Inducible Gene-I (RIG)-Like Receptor (RLR) Ligand.

13. The method of claim 8, wherein the immunological adjuvant is a Receptor for Advanced Glycation Endproducts (RAGE) Ligand.

14. The method of claim 9, wherein the immunological adjuvant is monophosphoryl lipid A (MPL).

15. The method of claim 9, wherein the immunological adjuvant is lipopolysaccharide (LPS).

16. The method of claim 8, wherein the immunological adjuvant is selected from the group consisting of LPS or derivatives thereof, CpG oligos, TLR3 ligands, TLR7 ligands, TLR9 ligands, MPL ligands, and RC529.

17. The method of claim 5, wherein the one or more therapeutic agents is a cancer antigen.

18. The method of claim 5, wherein the one or more therapeutic agents is selected from the group consisting of tumor antigens, CD4⁺ T-cell epitopes, cytokines, chemotherapeutic agents, radionuclides, small molecule signal transduction inhibitors, photothermal antennas, small interfering RNAs, monoclonal antibodies, and immunologic danger signaling molecules.

19. The method of claim 5, wherein the therapeutic agent is Sipuleucel-T.

20. The method of claim 5, wherein the therapeutic agent is carbonic anhydrase-IX.

21. The method of claim 5, wherein the therapeutic agent is carcinoembryonic antigen.

22. The method of claim 1, wherein the step of ablating the cancer is accomplished by a method selected from the group consisting of cryoablation, thermal ablation, radiotherapy, chemotherapy, radiofrequency ablation, electroporation, alcohol ablation, high intensity focused ultrasound, photodynamic therapy, monoclonal antibodies, and immunotoxins.

23. The method of claim 1, wherein the step of ablating the cancer is accomplished by cryoblation.

24. A method for treating an abnormal cellular proliferation in a patient comprising the steps of:
a. administering at or near the site of the abnormal cellular proliferation an effective amount of a composition that promotes a therapeutic immune response to the abnormal cellular proliferation comprising (i) a polymeric particle; and (ii) optionally one or more therapeutic agents encapsulated in or incorporated on or into the polymeric particle;
b. ablating the abnormal cellular proliferation.

25. The method of claim 24, further comprising administering an effective amount of an agent that mitigates suppression of anti-tumor immunity to the patient prior to or after administering the composition.

26. The method of claim 25, wherein the agent is selected from the group consisting of alkylating agents, steroids, nucleotide inhibitory drugs, chemotherapeutics, monoclonal antibodies, toxins, and inflammatory reducing agents.

27. The method of claim 25, wherein the agent is selected from the group consisting of cyclophosphamide, 5-fluorouracil, gemcitabine, doxorubicin, denileukin, diftitox, bevacizumab, and docetaxel.

28. The method of claim 24, wherein the polymeric particle comprises PLA, PGA, PLGA or co-polymers thereof.

29. The method of claim 24, wherein the polymeric particle is PLGA.

30. The method of claim 24, wherein the composition further comprises one or more immunological adjuvants encapsulated in or incorporated on or into the polymeric particle.

31. The method of claim 30, wherein the immunological adjuvant is a TLR Ligand.

32. The method of claim 30, wherein the immunological adjuvant is a C-Type Lectin Receptor Ligand.

33. The method of claim 30, wherein the immunological adjuvant is a NOD-Like Receptor Ligand.

34. The method of claim 30, wherein the immunological adjuvant is an RLR Ligand.

35. The method of claim 30, wherein the immunological adjuvant is a RAGE Ligand.

36. The method of claim 31, wherein the immunological adjuvant is monophosphoryl lipid A (MPL).

37. The method of claim 31, wherein the immunological adjuvant is lipopolysaccharide (LPS).

38. The method of claim 30, wherein the immunological adjuvant is selected from the group consisting of LPS or derivatives thereof, CpG oligos, TLR3 ligands, TLR7 ligands, TLR9 ligands, MPL ligands, and RC529.

39. The method of claim 24, wherein the one or more therapeutic agents is an antigen preferentially expressed by the abnormally proliferating cell.

40. The method of claim 24, wherein the one or more therapeutic agents is a cancer antigen.

41. The method of claim 24, wherein the one or more therapeutic agents is selected from the group consisting of tumor antigens, CD4⁺ T-cell epitopes, cytokines, chemotherapeutic agents, radionuclides, small molecule signal transduction inhibitors, photothermal antennas, small interfering RNAs, monoclonal antibodies, and immunologic danger signaling molecules.

42. The method of claim 24, wherein the therapeutic agent is Sipuleucel-T.

43. The method of claim 42, wherein the abnormal cellular proliferation is prostate cancer.

44. The method of claim 24, wherein the therapeutic agent is carbonic anhydrase-IX.

45. The method of claim 44, wherein the abnormal cellular proliferation is kidney cancer, colon cancer or cervical cancer.

46. The method of claim 24, wherein the therapeutic agent is carcinoembryonic antigen.

47. The method of claim 46, wherein the abnormal cellular proliferation is breast cancer, lung cancer or colon cancer.

48. The method of claim 24, wherein the step of ablating the cancer is accomplished by a method selected from the group consisting of cryoablation, thermal ablation, radiotherapy, chemotherapy, radiofrequency ablation, electroporation, alcohol ablation, high intensity focused ultrasound, photodynamic therapy, monoclonal antibodies, and immunotoxins.

49. The method of claim 24, wherein the step of ablating the cancer is accomplished by cryoblation.

50. A method for treating a solid tumor in a patient comprising the steps of:
a. administering an effective amount of an agent that mitigates suppression of anti-tumor immunity to the patient;
b. administering at or near the tumor site an effective amount of a composition comprising (i) a polymeric nanoparticle; (ii) one or more TLR ligands, C-Type Lectin Receptor ligands, NOD-Like Receptor Ligands, RLR Ligands, and/or RAGE Ligands encapsulated in or incorporated on or into the nanoparticle; and (iii) one or more tumor antigens encapsulated in the nanoparticle;
c. applying cryoablation to the solid tumor.

51. A method for treating a solid tumor in a patient comprising the steps of:
a. administering at or near the tumor site an effective amount of a composition comprising (i) a polymeric nanoparticle; (ii) one or more TLR ligands, C-Type Lectin Receptor ligands, NOD-Like Receptor ligands, RLR ligands, and/or RAGE ligands encapsulated in or incorporated on or into the nanoparticle; and (iii) one or more tumor antigens encapsulated in the nanoparticle;
b. ablating the solid tumor.

52. A method for treating a cancer in a patient comprising the steps of:
a. administering an effective amount of cyclophosphamide to the patient;
b. administering at or near the tumor site an effective amount of a composition comprising (i) a nanoparticle comprising PLGA; (ii) MPL incorporated on to the nanoparticle; and (iii) one or more tumor antigens encapsulated in the nanoparticle; and
c. ablating the cancer.
